(19) United States
(12) Patent Application Publication
(10) Pub. No.: US 2018/0344138 A1
(43) Pub. Date: Dec. 6, 2018

(54) ENDOSCOPE SYSTEM

(71) Applicant: OLYMPUS CORPORATION, Tokyo (JP)

(72) Inventor: Masahiro KUDO, Hino-shi (JP)

(73) Assignee: OLYMPUS CORPORATION, Tokyo (JP)

(21) Appl. No.: 16/059,360

(22) Filed: Aug. 9, 2018

Related U.S. Application Data
(63) Continuation of application No. PCT/JP2017/009563, filed on Mar. 9, 2017.

(30) Foreign Application Priority Data
Apr. 19, 2016 (JP) 2016-083796

(51) Int. Cl. A61B 1/045; G02B 23/24; A61B 1/00

(52) U.S. Cl. CPC A61B 1/045; A61B 1/0676; A61B 1/00009; G02B 23/24

(57) ABSTRACT

An endoscope system includes a video signal processing section configured to convert an endoscope image signal into a signal displayable on a display section, a status information notification necessity determination section configured to determine whether or not it is necessary to notify status information of a peripheral device, a visual line detection section configured to detect a visual line location of an operator in an endoscope image, an observation region setting section configured to set an observation region of the operator, a treatment instrument sensing section configured to sense a region in which a treatment instrument exists in the observation region, a status display control section configured to set a status display region in which the status information is displayed in a region within the observation region, and a status display superimposition section configured to superimpose the status information on the endoscope image in the status display region.

### CROSS REFERENCE TO RELATED APPLICATION

This application is a continuation application of PCT/JP2017/009563 filed on Mar. 9, 2017 and claims benefit of Japanese Application No. 2016-083796 filed in Japan on Apr. 19, 2016, the entire contents of which are incorporated herein by this reference.

### BACKGROUND OF THE INVENTION

### Field of the Invention

An embodiment of the present invention relates to an endoscope system and, more particularly, to an endoscope system in which peripheral device information can be displayed on an endoscope monitor in a superimposed manner,

### Description of the Related Art

Conventionally, in a medical field, endoscope apparatuses have been widely used for observation of organs in body cavity, remedial treatment using treatment instruments, surgical operations under endoscopic observation, and the like. In an endoscope apparatus, in general, an image pickup signal of an object captured by an electronic endoscope having an image pickup device such as a charge coupled device (CCD) mounted on a distal end of an insertion portion is transmitted to a processor and subjected to image processing. An endoscope image obtained through the image processing is outputted from the processor to an endoscope monitor and displayed on the endoscope monitor.

For remedial treatment or surgical operations under endoscopic observation, an endoscope system using such a type of endoscope apparatus and accompanying equipment including a light source apparatus, a processor, and an endoscope monitor, as well as a plurality of peripheral devices such as an insufflation device and an electrocautery device, is constructed and put to practical use.

Each of the peripheral devices has its own display means. In conventional endoscope systems, status information such as setting values, errors, and warnings regarding each device is displayed on the display means provided to each device. However, since the peripheral devices are dispersedly placed in an operating room, it is troublesome for an operator to check the display means of the peripheral devices individually, and the operator is prevented from smoothly carrying out a surgical operation.

On the other hand, an endoscope system that also displays status information of peripheral devices in a consolidated manner on an endoscope monitor has been proposed. An endoscope system has also been proposed that analyzes an endoscope image and, when detecting that a treatment instrument is coming close to an affected area, displays a warning message by superimposing the warning message on the endoscope image (for example, see Japanese Patent Application Laid-Open Publication No. 2011-212245).

In the proposals described above, since peripheral device information and warning messages are consolidated on an endoscope monitor, an operator can acquire necessary information from the endoscope monitor.

In the proposals, a display location of status information such as the peripheral device information or the warning message is a specified location (fixed location) provided on the endoscope monitor or in a vicinity of an affected area.

### SUMMARY OF THE INVENTION

An endoscope system according to an aspect of the present invention includes: a video signal processing section configured to convert an inputted endoscope image signal into a signal displayable on a display section; a status information notification necessity determination section configured to receive status information of a peripheral device and determine whether or not it is necessary to notify the status information to an operator; a visual line detection section configured to detect an observation location of the operator in an endoscope image by sensing a visual line of the operator; an observation region setting section configured to set an observation region of the operator based on a result of detection by the visual line detection section; a treatment instrument sensing section configured to sense, by image processing, a region in which a treatment instrument exists in the observation region; a status display control section configured to set a status display region in which the status information is displayed, in a region within the observation region excluding a display prohibition region, which is set around the observation location, and the region sensed by the treatment instrument sensing section, when the status information notification necessity determination section determines that it is necessary to notify the operator; and a status display superimposition section configured to superimpose, in the status display region, the status information on the signal outputted from the video signal processing section.

### BRIEF DESCRIPTION OF THE DRAWINGS

FIG. 1 is a diagram for describing an example of an entire configuration of an endoscope system according to an embodiment of the present invention;

FIG. 2 is a block diagram for describing an example of a configuration of an endoscope display image generation section;

FIG. 3 is a block diagram for describing an example of a configuration of a visual line detection section;

FIG. 4 is a flowchart for describing a procedure setting an observation region;

FIG. 5 is a flowchart for describing a procedure of detecting a forceps region;

FIG. 6 is a table for describing an example of display target status information and displayed contents;

FIG. 7 is a flowchart for describing a procedure of determining necessity or unnecessity of a status display;

FIG. 8 is a flowchart for describing a procedure of setting a status display location;

FIG. 9 is a flowchart for describing a procedure of generating an endoscope display image;

FIG. 10 is a diagram for describing an example of the status display location in the endoscope display image;

FIG. 11 is a diagram for describing an example of the endoscope display image with the superimposed status display;

FIG. 12 is a diagram for describing another example of the endoscope display image with the superimposed status display; and

FIG. 13 is a diagram for describing still another example of the endoscope display image with the superimposed status display.

### DETAILED DESCRIPTION OF THE PREFERRED EMBODIMENT(S)

Hereinafter, an embodiment will be described with reference to drawings.

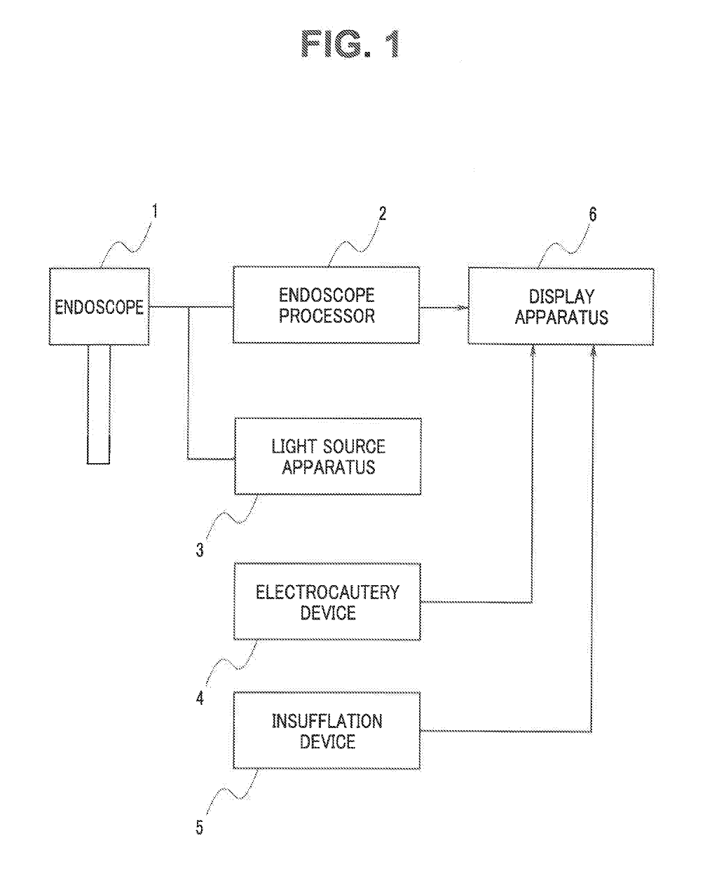

FIG. 1 is a diagram for describing an example of an entire configuration of an endoscope system according to an embodiment of the present invention. The endoscope system according to the present embodiment is used for, for example, an operation under endoscopic observation to treat, using treatment instruments such as an electrocautery, an affected area in a patient's abdominal cavity enlarged by feeding air such as carbon dioxide.

As FIG. 1 shows, the endoscope system includes an endoscope 1 configured to be inserted into a body cavity to observe or treat an affected area, an endoscope processor 2 configured to perform predetermined signal processing on a video signal of an image picked up by the endoscope 1, and a light source apparatus 3. A display apparatus 6 configured to display a signal-processed video is connected to the endoscope processor 2. The endoscope system also includes an electrocautery device 4 and an insufflation device 5 as peripheral devices required to treat an affected area. The electrocautery device 4 and the insufflation device 5 are connected to the display apparatus 6 and configured to be able to transmit various status information, which indicates settings, status, warnings, and errors regarding the devices. Note that the peripheral devices are not limited to the electrocautery device 4 and the insufflation device 5, but may include other devices required for operations such as an ultrasound coagulation dissection device.

The endoscope 1 includes an elongated insertion portion configured to be insertable into a body cavity or the like of a patient. An image pickup device such as a CCD is disposed on a distal end of the insertion portion. Note that the insertion portion may be flexible, or may be rigid (a rigid endoscope used for surgical operations). A light guide that guides illuminating light to the distal end of the insertion portion is also provided to the endoscope 1.

The endoscope processor 2 performs various processing on the video signal outputted from the image pickup device and generates an endoscope image to be displayed on the display apparatus 6. More specifically, the endoscope processor 2 performs predetermined processing, such as AGC (auto gain control) processing and CDS (correlated double sampling) processing, on the analog video signal outputted from the image pickup device, and then converts the analog video signal into a digital video signal. Thereafter, the endoscope processor 2 performs white balance processing, color correction processing, distortion correction processing, enhancement processing, and the like on the digital video signal and outputs the digital video signal to the display apparatus 6.

The light source apparatus 3 includes a light source, such as a lamp, that generates the illuminating light. The illuminating light radiated from the light source is collected to an entrance end face of the light guide of the endoscope 1. Note that other than the lamp, for example, a semiconductor light source typified by LED and laser diode may be used for the light source. In case of using the semiconductor light source, a semiconductor light source outputting white light may he used. Alternatively, semiconductor light sources may be provided for color components R (red), G (green), and B (blue), respectively, and white light may be obtained by mixing the respective color components of light outputted from the semiconductor light sources.

The display apparatus 6 includes an endoscope display image generation section 60 configured to generate an endoscope display image by superimposing, when necessary, the status information inputted from the electrocautery device 4 or the insufflation device 5 at a predetermined location on the endoscope image inputted from the endoscope processor 2, and a display section 68 configured to display the endoscope display image.

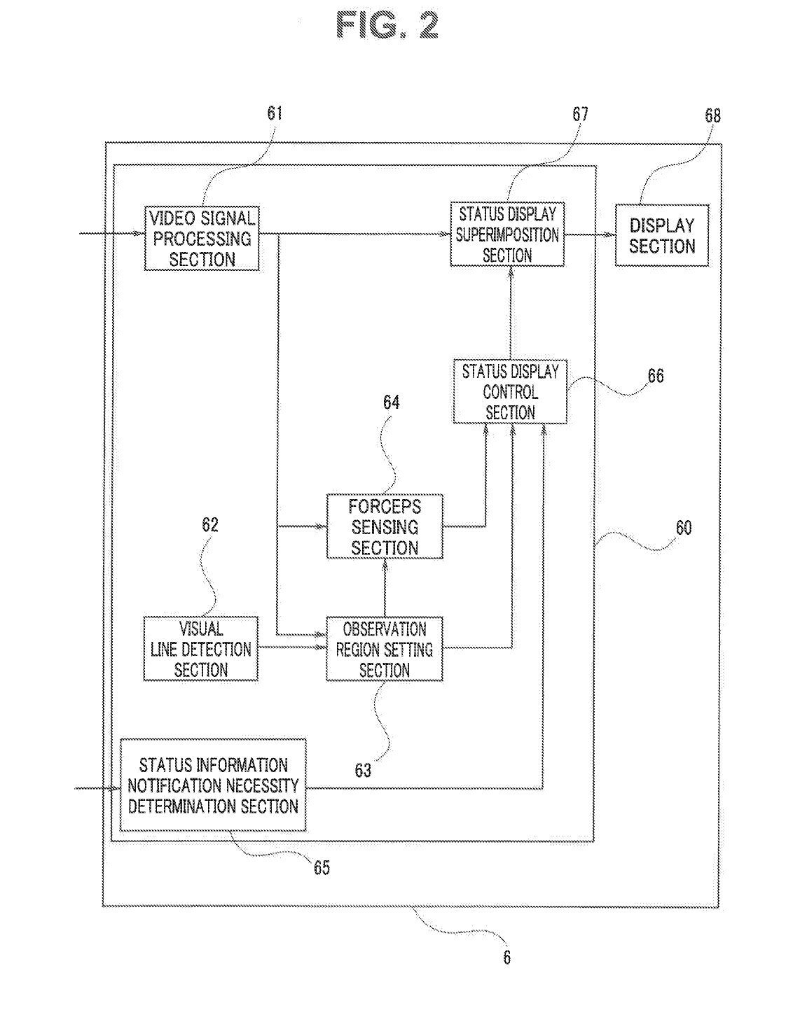

FIG. 2 is a block diagram for describing an example of a configuration of the endoscope display image generation section. As FIG. 2 shows, the endoscope display image generation section 60 includes a video signal processing section 61, a visual line detection section 62, an observation region setting section 63, and a forceps sensing section 64. The endoscope display image generation section 60 also includes a status information notification necessity determination section 65, a status display control section 66, and a status display superimposition section 67.

The video signal processing section 61 performs predetermined processing, such as converting the video signal inputted from the endoscope processor 2 into a signal format displayable on the display section 68.

The visual line detection section 62 detects a visual line location of an operator in the endoscope image. For the detection of the visual line location, a conventionally performed method (a method in which a visual line is detected by detecting a reference point and a movement point of an eye, and determining a location of the movement point relative to the reference point) can be used. A configuration of the visual line detection section 62 will be described in case of using the method in which a visual line direction is identified by detecting, for example, a location of a corneal reflex as the reference point and a location of a pupil as the movement point.

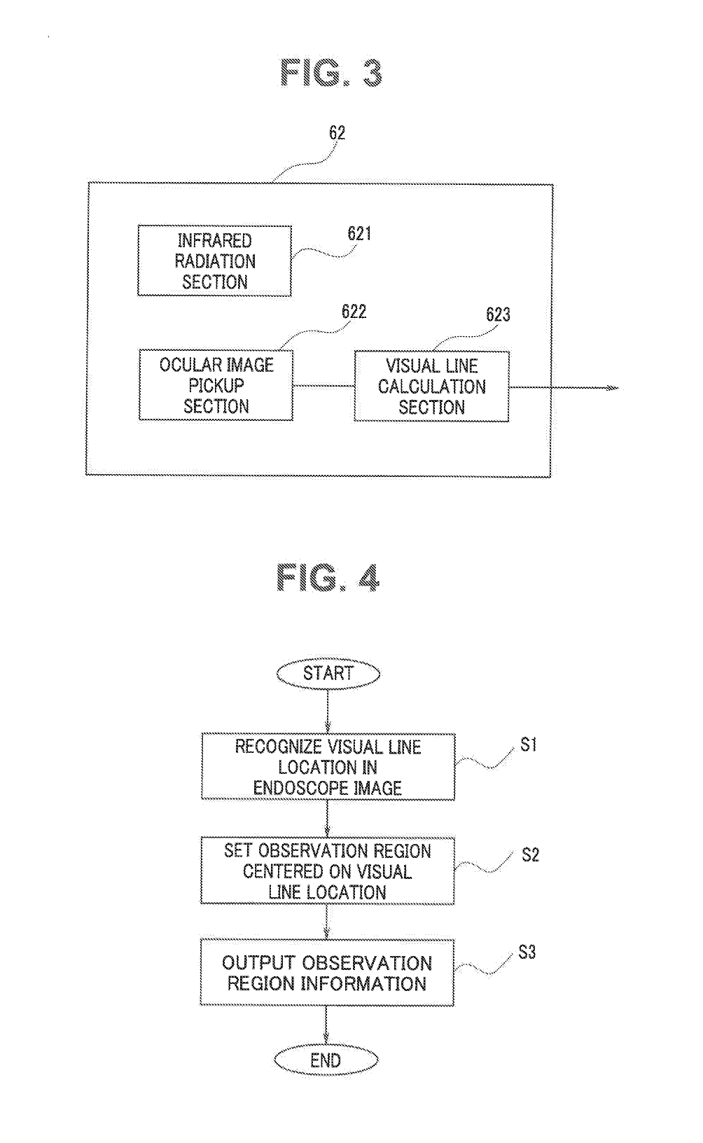

FIG. 3 is a block diagram for describing an example of the configuration of the visual line detection section. As FIG. 3 shows, the visual line detection section 62 includes an infrared radiation section 621, an ocular image pickup section 622, and a visual line calculation section 623. The infrared radiation section 621 includes, for example, an infrared LED and irradiates infrared rays toward a face of the operator. The ocular image pickup section 622 includes, for example, an infrared camera and obtains an ocular image by receiving light reflected from an eyeball of the operator by the irradiation of the infrared rays. The visual line calculation section 623 analyzes the ocular image and calculates a location of the reflected light on cornea (a location of a corneal reflex) and a location of a pupil, thereby identifying a visual line direction. The visual line calculation section 623 then calculates the visual line location of the operator in the endoscope image by using the visual line direction. In general, the visual line location is calculated as a coordinate location (xc, ye) in two-dimensional space with an x axis representing a horizontal direction of the endoscope image and a y axis representing a vertical direction of the endoscope image.

The observation region setting section 63 sets, in the endoscope image, a region in which the operator can instantly identify information (an observation region). FIG. 4 is a flowchart for describing a procedure of setting the observation region. First, the observation region setting section 63 recognizes the visual line location (xe, ye) in the endoscope image, inputted from the visual line detection section 62 (step S1). Next, the observation region setting section 63 sets the observation region centered on the visual line location in the endoscope image inputted from the video signal processing section 61, by using various information including horizontal and vertical sizes of the display section 68, a distance from the operator to the display section 68, and a visual field range within which the operator can instantly identify information (for example, a discrimination visual field, which is a visual field range within which a human being can recognize an object in detail without moving eyeballs: a visual field range at 5 degrees in each of the horizontal and vertical directions with respect to the visual line direction) (step S2). The distance from the operator to the display section 68 can be obtained by selecting one of distances under a practical use condition by using setting means (not shown), or by measuring the distance by providing two of the ocular image pickup section 62.2 included in the visual line detection section 62. Finally, the observation region setting section 63 outputs the set observation region to the forceps sensing section 64 and the status display control section 66 (step S3). In such a manner, the observation region setting section 63 sets the observation region centered on the visual line location (xe, ye) in the endoscope image.

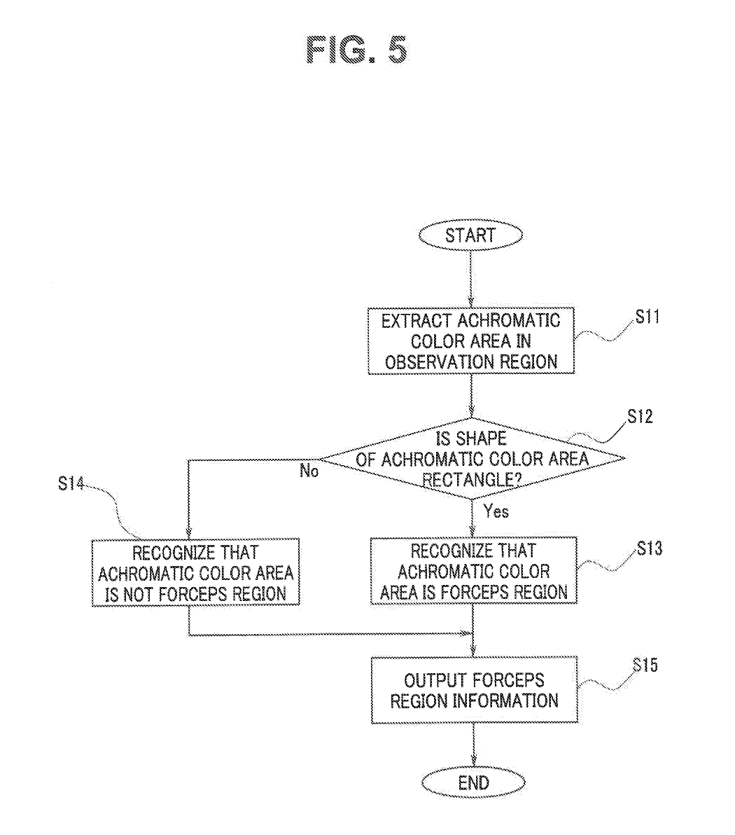

The forceps sensing section 64 determines whether or not forceps exist in the observation region and, when forceps exist in the observation region, identifies where the forceps are (a forceps region). FIG. 5 is a flowchart for describing a procedure of detecting the forceps region. First, the forceps sensing section 64 identifies the observation region inputted from the observation region setting section 63 in the endoscope image inputted from the video signal processing section 61.

Then, in the observation region, the forceps sensing section 64 extracts an achromatic color area (step S11). Next, the forceps sensing section 64 identifies a shape of the extracted achromatic color area. If the shape of the achromatic color area is an approximate rectangle (step S12; Yes), the forceps sensing section 64 recognizes that the achromatic color area is the forceps region (step S13). If the shape of the achromatic color area is another shape than an approximate rectangle (step S12; No), the forceps sensing section 64 recognizes that the achromatic color area is not the forceps region (step S14). Finally, the forceps sensing section 64 outputs the forceps region identified within the observation region to the status display control section 66 (step S15).

Note that if a plurality of achromatic color areas exist in the observation region, shapes of all of the achromatic color areas are identified. In the above-described example, forceps are gray (silver) to black in color and have linear appearances while most of surfaces in a body cavity (human tissue) are dark red to orange in color and have curved appearances. The forceps region is extracted by taking note of such color (chroma) and shape differences. However, the forceps region may be extracted by using other methods.

The status information notification necessity determination section 65 determines whether or not it is necessary to display the status information inputted from any one of the peripheral devices on the endoscope image in a superimposed manner. In general, various peripheral devices are connected to the endoscope system, and wide-ranging information is outputted from the peripheral devices. However, if all of such information is displayed on the display apparatus 6, it is feared that essential information is buried in other information and overlooked, or that a displayed content is changed so frequently that the operator cannot concentrate on a procedure. Accordingly, of the information outputted from the peripheral devices, high-priority information required for the operator to perform the procedure with is preset, and only the preset status information is extracted and displayed along with the endoscope image on the display apparatus 6.

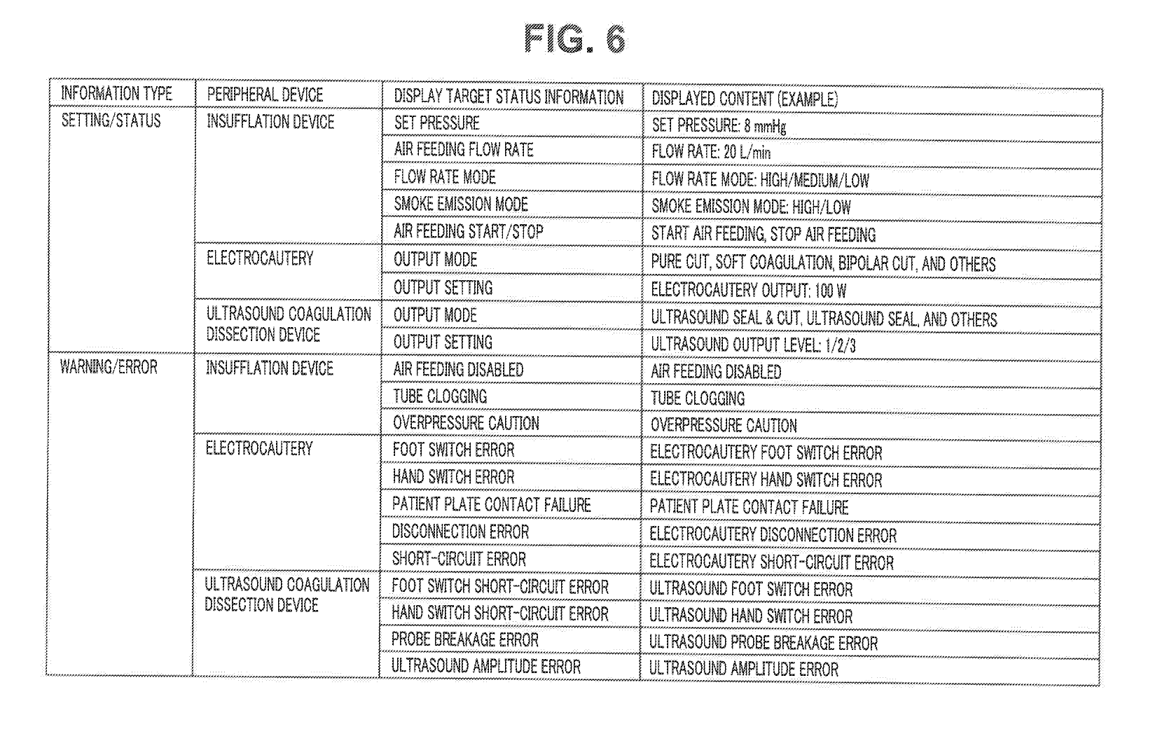

FIG. 6 is a table for describing an example of display target status information and displayed contents. The status information is broadly categorized into information on settings and status of the peripheral devices, and information on warnings and errors. For each of information types, the status information to be displayed on the display apparatus 6 (display target status information) and displayed contents to be displayed when such status information is inputted are set prior to an operation, for each peripheral device.

As FIG. 6 shows, for example, in case of the insufflation device, the status information on each of items “set pressure”, “air feeding flow rate”, “flow rate mode”, “smoke emission mode”, and “air feeding start/stop” is set as the display target status information with respect to “setting/status”. With respect to “warning/error”, the status information on each of alarmed matters “air feeding disabled”, “tube clogging”, and “overpressure caution” is set as the display target status information. The display target status information is also set for each of the electrocautery device, the ultrasound coagulation dissection device, and other necessary peripheral devices similarly to the insufflation device.

The status information notification necessity determination section 65 determines whether or not to allow the display apparatus 6 to display the status information inputted from any one of the peripheral devices, by referring to the preset display target, status information.

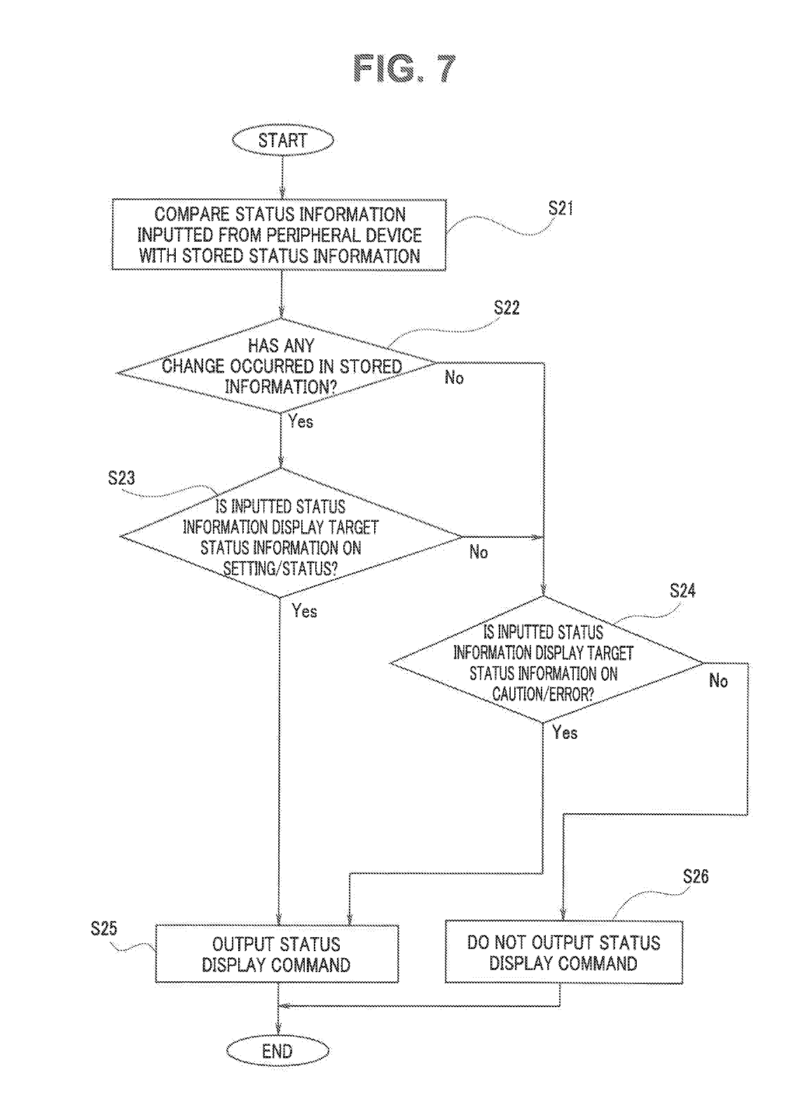

FIG. 7 is a flowchart for describing a procedure of determining whether a status display is needed or not. First, the status information notification necessity determination section 65 compares the status information inputted from any one of the peripheral devices with the stored status information (step S21). The status information is inputted from the peripheral device to the display apparatus 6 in real-time (or at a constant interval). For the inputted status information, the latest (most recent) content is stored in a memory or the like (not shown). In step S21, for the status information inputted from the peripheral device, the status information stored in the memory or the like and the inputted status information are compared. For example, when the status information on “set pressure” is inputted from the insufflation device 5, a most recent value of the set pressure of the insufflation device 5 stored in the memory or the like and an inputted value of the set pressure are compared.

If the inputted status information is different from the stored status information (step S22; Yes), the status information notification necessity determination section 65 determines whether or not the inputted status information applies to the display target status information on setting/status (step S23). For example, in step S22, when the status information indicating that the set pressure is 8 mmHg is inputted from the insufflation device 5 and the stored most recent set pressure of the insufflation device 5 is 6 mmHg, the status information notification necessity determination section 65 determines that the inputted status information is different from the stored status information.

If the inputted status information applies to the display target status information on setting/status (step S23; Yes), the status information notification necessity determination section 65 determines that it is necessary to display the inputted status information, and outputs a status display command (step S25).

On the other hand, if the inputted status information does not apply to the display target status information on setting/status (step S23; No), the status information notification necessity determination section 65 determines whether or not the inputted status information applies to the display target status information on warning/error (step S24). Note that when determining that the inputted status information is equal to the stored status information (step S22; No), the status information notification necessity determination section 65 also proceeds to step S24 and determines whether or not the inputted status information applies to the display target status information on warning/error.

If the inputted status information applies to the display target status information on warning/error (step S24; Yes), the status information notification necessity determination section 65 determines that it is necessary to display the inputted status information, and outputs a status display command (step S25). The status information notification necessity determination section 65 also outputs one of the displayed contents corresponding to the status information along with the status display command. On the other hand, if the inputted status information does not apply to the display target status information on warning/error (step S24; No), the status information notification necessity determination section 65 determines that it is unnecessary to display the inputted status information, and does not output a status display command (step S26).

Note that if multiple pieces of the status information are concurrently inputted from any of the peripheral devices, the status information notification necessity determination section 65 performs a series of the processing from steps S21 to S26 shown in FIG. 7 to determine whether or not to output a status display command, for each piece of the status information individually.

For example, if the status information indicating that the set pressure is 8 mmHg is inputted from the insufflation device 5 and concurrently a warning of disconnection error is inputted from the electrocautery device 4, the status information notification necessity determination section 65 determines whether it is necessary to display the status information on the set pressure of the insufflation device 5, and also determines whether it is necessary to display the status information on the warning of disconnection error of the electrocautery device 4.

For example, if the set pressure of the insufflation device 5 has not changed from the stored most recent value and if the warning of disconnection error of the electrocautery device 4 is continuously inputted, the status information notification necessity determination section 65 determines that it is unnecessary to display the set pressure of the insufflation device 5, and determines that it is necessary to display the warning of disconnection error of the electrocautery device 4. Accordingly, in this case, the status information notification necessity determination section 65 outputs a status display command only for the warning of disconnection error of the electrocautery device 4.

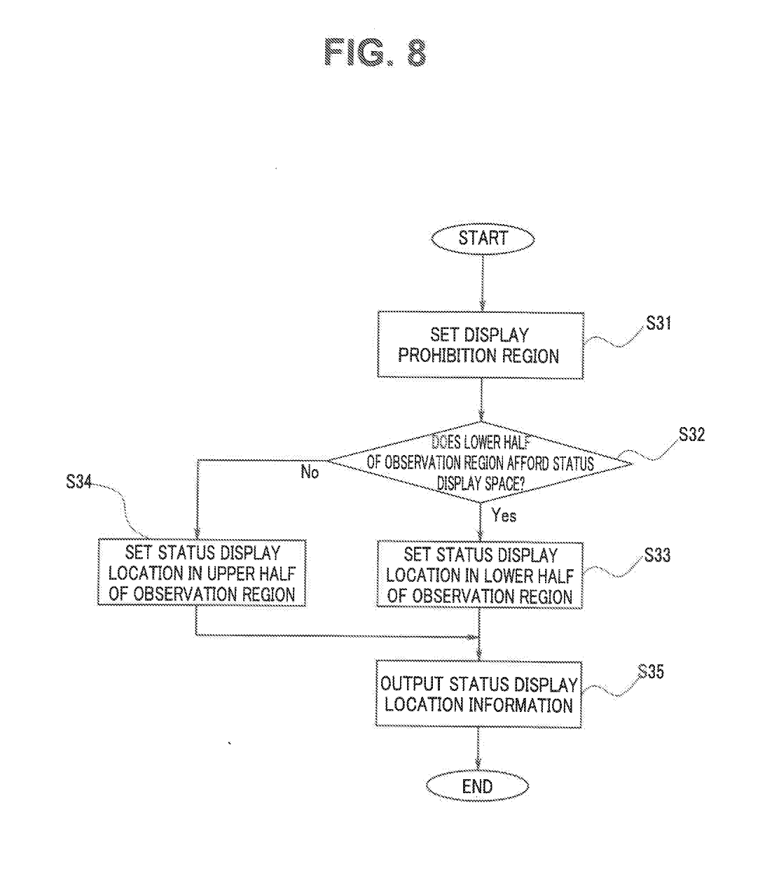

The status display control section 66 sets a display location of the status information to be superimposed on the endoscope image. Then, when the status display command is inputted from the status information notification necessity determination section 65, the status display control section 66 outputs the display location and the displayed content to the status display superimposition section 67. FIG. 8 is a flowchart for describing a procedure of setting the status display location. First, in the observation region inputted from the observation region setting section 63, the status display control section 66 sets a region in which the status information must not be displayed due to a possibility of interrupting the procedure (hereinafter, referred to as a “display prohibition region”) (step S31). For example, the status display control section 66 divides the observation region into three equal areas in the horizontal direction, and further divides each of the three equal areas into three equal areas in the vertical direction to obtain nine areas. Of the nine areas, the status display control section 66 sets a center area, which includes the visual line location, as the display prohibition region.

Next, the status display control section 66 divides the observation region into two equal areas in the vertical direction, and determines whether or not the lower half of the areas affords a space capable of displaying the status information (step S32). In general, when a human being moves a visual line upward or downward, moving the visual line downward causes a smaller burden on eyes than moving the visual line upward. Accordingly, the status display control section 66 first searches the lower half area of the observation region for the space capable of displaying the status information. In the lower half area of the observation region excluding the display prohibition region set in step S31 and the forceps region inputted from the forceps sensing section 64, the status display control section 66 identifies a region in which the status information can be displayed. Then, in the identified region, the status display control section 66 determines whether or not the space in which a status display region of a preset size is disposed exists.

If it is determined that the lower half area of the observation region affords the space capable of displaying the status information (step S32; Yes), the status display control section 66 sets a status information display location within the identified region (step S33). The status information display location is preferably a location that makes the operator move the visual line rightward and leftward as little as possible and hardly interferes with the visual line location at which the operator is gazing. Accordingly, for example, a location that is closest to the visual line location horizontally and is closest to an edge of the observation region vertically is set as the status information display location.

On the other hand, if it is determined that the lower half area of the observation region does not afford the space capable of displaying the status information (step S32; No), the status display control section 66 sets a status information display location in the upper half area of the observation region (step S34). As in the case of being set in the lower half area of the observation region, the status information display location is preferably a location that makes the operator move the visual line rightward and leftward as little as possible and hardly interferes with the visual line location at which the operator is gazing. Accordingly, for example, a location that is closest to the visual line location horizontally and is closest to an edge of the observation region vertically is set as the status information display location.

Finally, the status display control section 66 outputs the status information display location set in step S33 or S34 (step S35).

When the displayed content of the status information and the status information display location are inputted from the status display control section 66 the status display superimposition section 67 superimposes a status display on the endoscope image inputted from the video signal processing section 61 and generates and outputs an endoscope display image. Note that if no input is received from the status display control section 67, the status display superimposition section 67 outputs the endoscope image inputted from the video signal processing section 61 as it is, as the endoscope display image.

The display section 68 displays the endoscope display image inputted from the status display superimposition section 67.

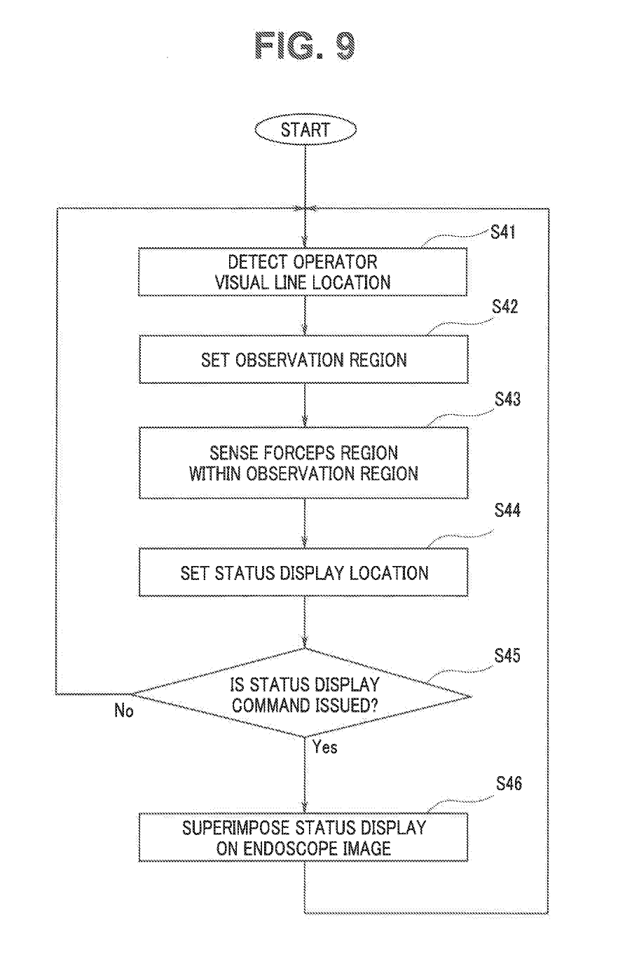

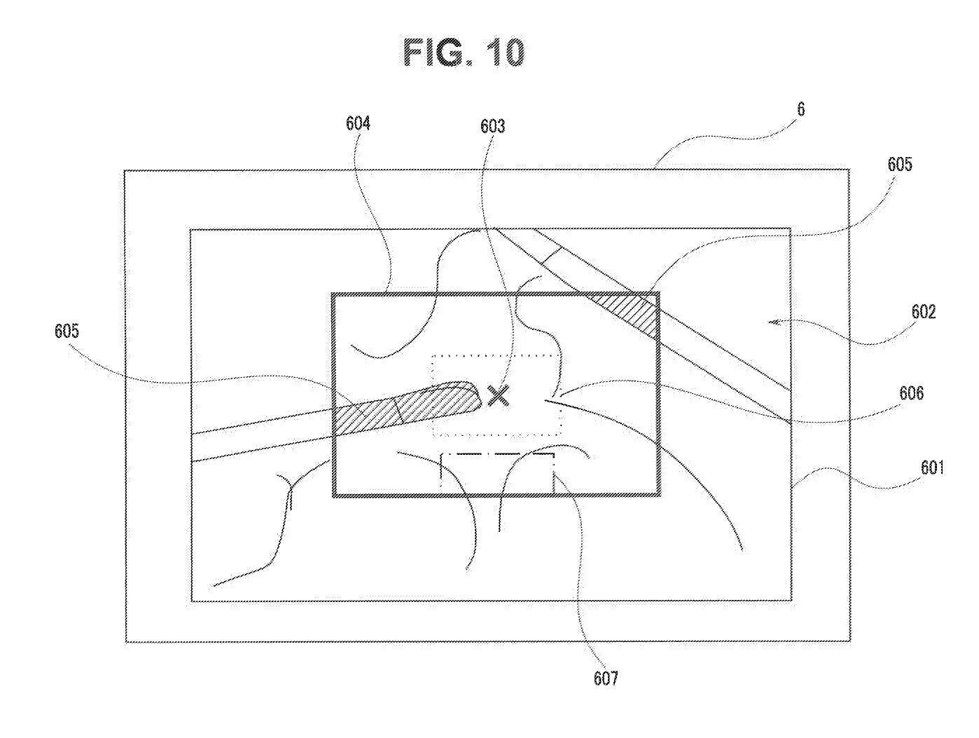

A description will be given of a series of procedures, in the endoscope display image generation section 60, of generating the endoscope display image to be displayed on the display section 68 based on the endoscope image inputted from the endoscope processor 2, with reference to FIGS. 9 and 10. FIG. 9 is a flowchart for describing the procedure of generating the endoscope display image, and FIG. 10 is a diagram for describing an example of the status display location in the endoscope display image.

First, the visual line detection section 62 detects the visual line location of the operator in the endoscope image inputted to the video signal processing section 61 (step S41). Next, the observation region setting section 63 sets the observation region in the endoscope image (step S42). More specifically, the observation region setting section 63 sets the observation region by performing a series of the procedure shown in FIG. 4. For example, in FIG. 10, when a visual line location 603 is a location denoted by “x”, a region of an approximate short rectangular shape enclosed by a thick line is set as an observation region 604.

Next, the forceps sensing section 64 senses the forceps region in the observation region (step S43). More specifically, the forceps sensing section 64 sets the forceps region by performing a series of the procedure shown in FIG. 5. For example, in FIG. 10, regions shaded with diagonal lines (two regions, one of which is in the middle of the left side of the observation region, and the other of which is in the upper right corner of the observation region) are set as forceps regions 605.

Subsequently, the status display control section 66 sets the status display location (step S44). More specifically, the status display control section 66 sets the status display location by performing a series of the procedure shown in FIG. 8. For example, in FIG. 10, in the lower half area of the observation region excluding a display prohibition region 606 (a region of an approximate short rectangular shape enclosed by a dotted line) and the forceps regions 605, a region in which a status display can be made exists. Accordingly, a status display location 607 is set at a location of an approximate short rectangular region enclosed by a dot-and-dash line.

Next, the status display control section 66 determines whether or not the status display command is inputted from the status information notification necessity determination section 65 (step S44). If the status display command is inputted (step S44; Yes), the status display superimposition section 67 superimposes the displayed content of the status information inputted from the status display control section 66, at the status display location (the status display location set in step 844), on the endoscope image inputted from video signal processing section 61, and generates and outputs the endoscope display image to the display section 68. Thereafter, process goes back to step S41, and a next endoscope display image is generated.

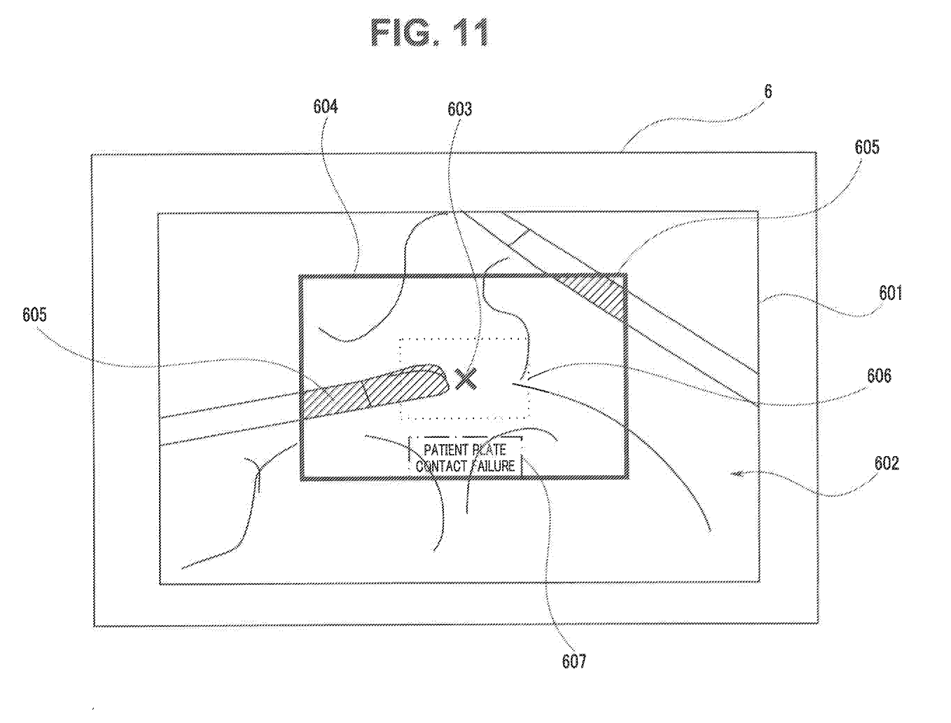

FIGS. 11, 12, and 13 are diagrams for describing examples of the endoscope display image with the superimposed status display. FIG. 11 shows an example of the endoscope display image in a case where an error of patient plate contact failure is inputted as the status information to the status information notification necessity determination section 65 from the electrocautery device 4, which is one of the peripheral devices.

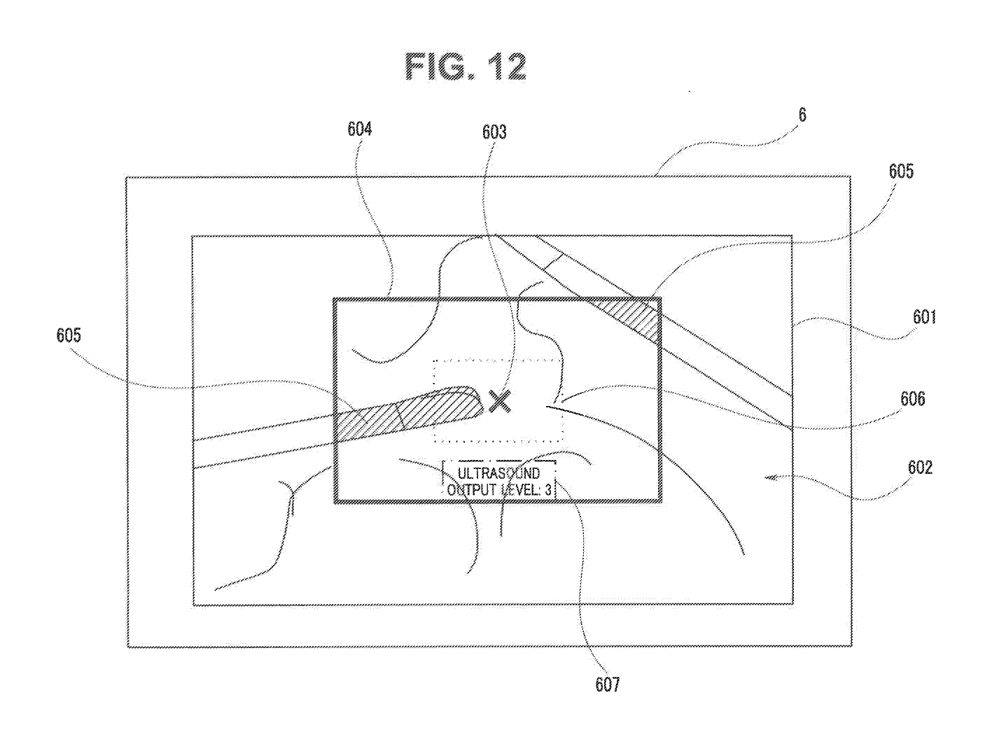

FIG. 12 shows an example of the endoscope display image in a case where the status information indicating that the ultrasound output level is 3 is inputted to the status information notification necessity determination section 65 from the ultrasound coagulation dissection device, which is one of the peripheral devices. Note that the status information is displayed as shown in FIG. 12 when the ultrasound output level has changed to 3 from a value other than 3, but the status information is not displayed when the output level is maintained at 3.

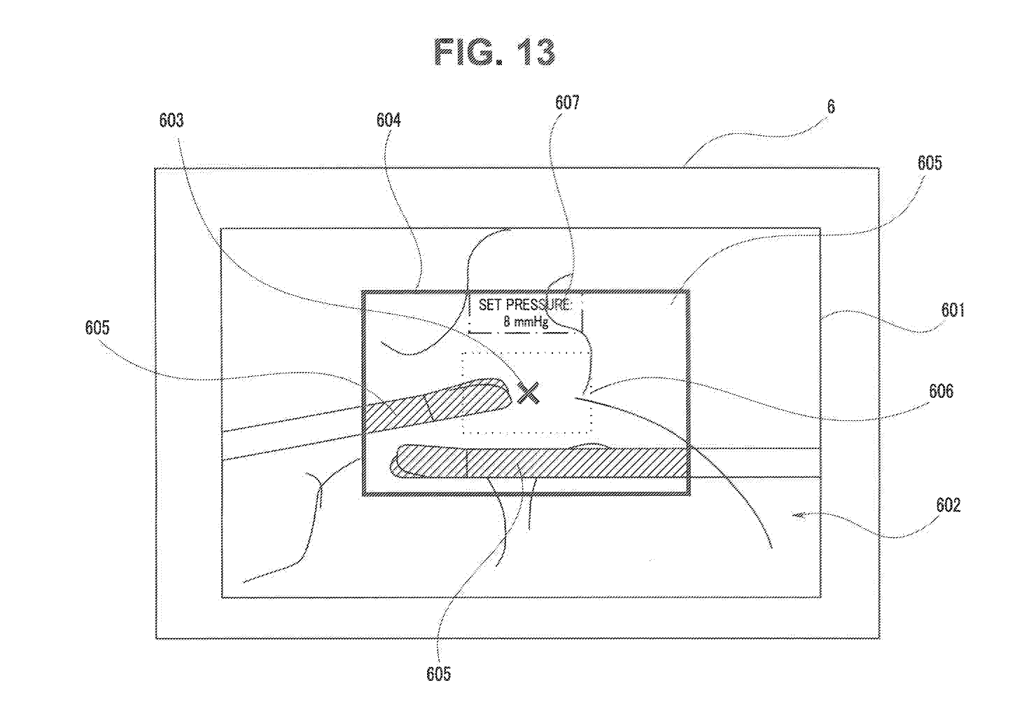

FIG. 13 shows an example of the endoscope display image in a case where the status information indicating that the set pressure is 8 mmHg is inputted to the status information notification necessity determination section 65 from the insufflation device 5, which is one of the peripheral devices. FIG. 13 shows a case where the status display location is set in the upper half area of the observation region because the status display region cannot be secured due to the forceps regions in the lower half area of the observation region. Note that the status information is displayed as shown in FIG. 13 when the set pressure of the insufflation device 5 has changed to 8 mmHg from a value other than 8 mmHg, but the status information is not displayed when the set pressure is maintained at 8 mmHg.

On the other hand, if the status display command is not inputted (step S44; No), the status display superimposition section. 67 outputs the endoscope image inputted from the video signal processing section 61 as it is, as the endoscope display image, to the display section 68. Then, process goes back to step S41, and a next endoscope display image is generated.

As described above, according to the present embodiment, when the status information such a setting, a status, or a warning message is inputted from any one of the peripheral devices, it is determined whether or not the inputted status information is the preset display target status information. If the inputted status information is the display target status information, the visual field range within which the operator can instantly identify information (the observation region) is identified in the endoscope image, the status display location is set in the observation region excluding the forceps region, and the status information is displayed. Accordingly, the status information of a peripheral device can be displayed in a superimposed manner on the endoscope image without lowering visibility.

Note that in a case where the status information inputted from a peripheral device is information on setting/status, although the status information is configured to be displayed only when a set value or a status has changed, the status information may be configured to be continuously displayed for a time period desired by the operator, by setting the time period for displaying the status information by using a timer or the like.

In the above description, the status information notification necessity determination section 65 determines whether or not to superimpose and display the status information on the endoscope image, and if it is determined that it is necessary to display the statue information, only such status information is configured to be automatically displayed. However, a configuration is also possible in which a status information display button or the like is provided, and the status information, in addition to being automatically displayed, is displayed at a timing desired by the operator.

Further, in the above description, although the endoscope display image generation section 60 is provided in the display apparatus 6, a configuration is also possible in which the endoscope display image generation section 60 is provided in the endoscope processor 2.

In the present description, each “section” is a conceptual component corresponding to each of functions of the embodiment and does not necessarily make a one-to-one correspondence to a specific piece of hardware or a software routine. Accordingly, in the present description, the embodiment is described, supposing virtual circuit blocks (sections) that have the individual functions of the embodiment, respectively. Each of the steps in each of the procedures in the embodiment, unless contrary to the nature of each step in each procedure, may be performed in a changed order, may be performed concurrently with another step or other steps, or may be performed in a different order each time. Further, all or part of the steps in the procedures of the embodiment may be implemented by hardware.

The embodiment of the present invention has been described. However, the embodiment is presented as an illustrative purpose and is not intended to limit the scope of the invention. The novel embodiment can be implemented in other various forms, and various omissions, replacements, and changes can be made without departing from the gist of the invention. The embodiment and modifications of the embodiment are included in the scope and gist of the invention, and also included in the inventions according to claims and the equivalent scopes of the inventions.

What is claimed is:

1. An endoscope system, comprising:
a video signal processing section configured to convert an inputted endoscope image signal into a signal displayable on a display section;
a status information notification necessity determination section configured to receive status information of a peripheral device and determine whether or not it is necessary to notify the status information to an operator;
a visual line detection section configured to detect an observation location of the operator in an endoscope image by sensing a visual line of the operator;
an observation region setting section configured to set an observation region of the operator based on a result of detection by the visual line detection section;
a treatment instrument sensing section configured to sense, by image processing, a region in which a treatment instrument exists in the observation region;
a status display control section configured to set a status display region in which the status information is displayed, in a region within the observation region excluding a display prohibition region, which is set around the observation location, and the region sensed by the treatment instrument sensing section, when the status information notification necessity determination section determines that it is necessary to notify the operator; and
a status display superimposition section configured to superimpose, in the status display region, the status information on the signal outputted from the video signal processing section.

2. The endoscope system according to claim 1, wherein the status display region is disposed in a vicinity of the display prohibition region.

3. The endoscope system according to claim 1, wherein when the status display region can be set in a lower half area of the observation region, the status display control section sets the status display region at a location that is at an edge of the lower half area of the observation region and is closest to the observation location horizontally.

4. The endoscope system according to claim 1, wherein when the status display region cannot be set in a lower half area of the observation region, the status display control section sets the status display region at a location that is at an edge of an upper half area of the observation region and is closest to the observation location horizontally.

5. The endoscope system according to claim 1, wherein when the status information inputted from the peripheral device is a warning or an alarm, the status information is continuously displayed in the status display region while the status information is inputted.
